(19) United States
(12) Patent Application Publication
(10) Pub. No.: US 2007/0237838 A1
(43) Pub. Date: Oct. 11, 2007

(54) A Herbal Composition for Treating Diseases and a Method for Obtaining the Same

(76) Inventor: Audino Mansilla, San Martinde Los Andes (AR)

Correspondence Address: NORRIS, MCLAUGHLIN & MARCUS, P.A., 875 THIRD AVE, 18TH FLOOR, NEW YORK, NY, 10022, US

(21) Appl. No.: 11/278,683

(22) Filed: Apr. 5, 2006

(51) Int. Cl. A61K 36/282; A61K 36/75

(52) U.S. Cl. 424/725; 424/740

(57) ABSTRACT

A natural herbal composition for the treatment of cancer, hepatitis C, HIV/AIDS, and Pengifoide Buloso, with the composition comprising a mixture of herbs Artemisa absinthium L. and Ruta graveolens L. in an infusion. A method for preparing the composition is also provided.

### BACKGROUND OF THE INVENTION

1. Field of the Invention

The present invention relates to the medical field and preferably to the homeopathic medical field and more particularly refers to a composition and a method to prepare the same, wherein the composition is based on herbs, namely Artemisa absinthium L. and Ruta graveolens L., mixed in a solvent, for treating diseases such as cancer, hepatitis C, HIV/AIDS and Pengifoide buloso.

2. Description of the Prior Art

Since 1998 the difference in the number of cancer cases between man and woman has been declining. In the first years, the disease appeared with most frequency in men, but in the last three years the number of cases in women has been increasing.

Regarding another disease, the 70% of the cases of hepatitis C correspond to men and, regarding HIV, the masculine population prevails in about a 65%.

As to the age of the patients, the average age is about 40/75 years in cancer cases, between 40/60 years for Hepatitis C, near 63 years in the case of Pengifoide Buloso and a range of 24/55 years for HIV.

While many traditional pharmaceuticals have been developed for treating all the above diseases no natural compositions, based on natural herbs, have been developed for such afflictions. However, it is well know the beneficial therapeutic effect of many herbs in connection to several diseases other than the above mentioned. The herb Artemisa absinthium, for example, commonly known in Latin America under the name “ajenjo”, is bush type plant growing as a wild plant in Europe, North Africa and west Asia. Actually this plant is also cultivated in North America. The leaves and flowers, as well as the oil obtained therefrom are used in herbalist. It is an herb that reaches a height of from 50 to 80 cm. and it has fuzz that gives a silver aspect to the plant. It also has clusters of yellow flowers.

The ajenjo has been used from ancient times for treating many pathologies. As a medicine, the ajenjo has been traditionally employed as a bitter agent for improving the digestion, for the treatment of worms and parasites and it is a stimulant for menstruation. It is considered as a useful remedy for liver and gall gladder afflictions. The most important compound of the plant is a volatile oil, which is obtained by distillation and normally the plant provides between 0, 5 and 1, 0% of oil. Usually, it has dark green color or sometimes blue, and has a strong aroma and a bitter flavor. This oil contains tuyona (absinthol or tenacetona), tuyilol (alone or combined with malic and isovalerianic acids), iso-tuyona, cadinena, felandreno and pineno. A very low content of this oil is present in the ajenjo tea. The oil also presents strong bitter agents as absintina and anabsintina, which acts as a stimulus for the digestive function.

Another well know beneficial plant or herb is the Ruda which is a perennial aromatic plant reaching a height of about 150 cm. It belongs to the family of Rutaceas and its scientific name is Ruta graveolens L. It possesses erect stems with ramifications and yellow-green meaty leaves, which have glands that provide to the plant its particular aroma and possesses 2 cm flowers with dentate petals. The fruit is in a capsule and the plant usually grows in dry Mediterranean lands.

The main important active principles of this herb are:

An essential oil rich in acids (anisic, caprilic and salicylic) terpenos (limonene, pinene y cineol)

undecanone, metilnonilcetone, metilnonil-carbinol

Alkaloids: arborinine, graveoline, graveolinine, skiaminine, dictamnine, citisine o soforine, cocusaginine, etc.

Tannins

cumarines, specially furocumarines, like bergaptene

Rutine.

Vitamin C.

The Ruda is employed as a protector of arteries and capillaries. This characteristic makes Ruda useful for the treatment of several diseases of the circulatory system like varicose veins, circulatory problems, etc. It is also used as a diluted infusion for the treatment of digestive problems.

Another characteristic of the Ruda is that the same is capable of inducing menstruation. This characteristic is based in some toxically compounds found in any ruda preparation like arborinine, graveoline, graveolinine, citisine or metilnonilcetone. These compounds enhance the blood circulation in the uterus, facilitating the menstruation.

The Ruda also has external applications in the field of the homeopathic medicine. Act as an analgesic reducing pain and inflammations in cases of blows, external injuries, sprains, etc.

As stated above, there are several compositions and formulations employing herbs and natural components for treating several diseases. U.S. Pat. No 6,696,094 discloses a pharmaceutical composition for treating patients affected by HIV/AIDS, wherein the pharmaceutical composition is in the form of an intravenous injection solution and optionally capsules. The pharmaceutical composition contains fourteen (14) ingredients, i.e., diffuse hedyotis, bistort rhizome, giant knotweed rhizome, Asiatic moonseed rhizome, baical skullcap root, bovine biliary powder, milkvetch root, barbary wolfberry fruit, sanqi, figwort root, Chinese magnoliavine fruit, turmeric root-tuber, hawthorn fruit, and Chinese angelica.

U.S. Pat. No. 6,649,650 relates to a novel synergistic composition of lignans exhibiting anticancer activities for breast, cervix, neuroblastoma, colon, liver, lung, mouth, ovary and prostate cancer obtained from the plant extract of Cedrus deodra,; further, the synergistic composition of lignan is used in combination with pharmaceutically acceptable carriers for inhibiting growth of various human cancer cell lines selected from breast, cervix, neuroblastoma, colon, liver, lung, mouth, ovary and prostate tissues.

U.S. Pat. No. 6,967,034 refers to a new herbal-based composition and method for treatment of CD33+ acute and chronic myeloid leukemia by Piper betel leaf extracts, provides a process for the isolation of active fractions from leaves or any other plant parts of Piper betel to treat CD33+ AML and CML with a simplified method of isolation of active components from all plant parts of Piper betel possessing biological activities relevant to the treatment of CD33+ AML and CML.

Considering that the therapeutic benefits of the above herbs seem to be not well demonstrated and that the herbs per se have not been widely employed in the treatment of such and other diseases and that there are still the need of having an effective formulation or composition based on natural components, the inventor has worked in the development of new and inventive alternatives.

### SUMMARY OF THE INVENTION

It is therefore an object of the present invention to provide a new herbal formulation for the treatment of HIV/AIDS, cancer, hepatitis C and Pengifoide buloso, with effective therapeutic results.

It is another object of the present invention to provide a natural herbal composition or formulation for the treatment of cancer, hepatitis C, HIV/AIDS, and Pengifoide Buloso, with the composition comprising a mixture of herbs Artemisa absinthium L. and Ruta graveolens L. in an infusion.

It is still another object of the present invention to provide an herbal composition or formulation for the treatment of a patient afflicted of at least one of the following diseases, HIV/AIDS, cancer, hepatitis C and Pengifoide buloso, the formulation comprising:

a mixture of herbs Artemisa absinthium L. and Ruta graveolens L. forming an infusion mixture in a solvent, wherein the solvent is water and preferably mineral water.

It is a further object of the present invention to provide an herbal composition or formulation for the treatment of a patient afflicted of at least one of the following diseases, HIV/AIDS, cancer, hepatitis C and Pengifoide buloso, the formulation comprising:

0.85 grams of said Artemisa absinthium L. and 1.15 grams of said Ruta graveolens L. per 1.5 liter of the mineral water.

It is even another object of the present invention to provide a method for preparing an herbal composition or formulation for the treatment of a patient afflicted of at least one of the following diseases, HIV/AIDS, cancer, hepatitis C and Pengifoide buloso, wherein the formulation comprises:

a mixture of herbs Artemisa absinthium L. and Ruta graveolens L. forming an infusion mixture in a solvent,

the method comprising the steps of:

i) mixing the Artemisa absinthium L. and the Ruta graveolens L. in the solvent at 100° C. for 15 seconds, to form a mixture and in a proportion equivalent to 0.85 grams of the Artemisa absinthium L., 1.15 grams of the Ruta graveolens L. and 1.5 liters of the solvent, ii) permitting the mixture to rest for obtaining an infusion and decanting the herbs, and

iii) filtering the infusion to obtain a filtered solution.

### DESCRIPTION OF THE PREFERRED EMBODIMENTS

Now referring in detail to the invention, the same refers to a herbal composition or formulation for the treatment of a patient in need thereof, wherein the patient, animal or a human being, is afflicted of a disease such as HIV/AIDS, cancer, hepatitis C and Pengifoide buloso, or a combination of same. The inventive composition comprises a mixture of herbs Artemisa absinthium L. and Ruta graveolens L. and preferably the mixture is in the form of an infusion mixture in a solvent such as water and preferably mineral water.

The best results have been obtained with mineral water and preferably a mineral water having the following characteristics:

Biochemical analysis of the water:

Bacteriological Analysis

Aerobic bacterial account

Grown at 37° C. for 24 hs on Nutritive agar: 2 cfu/mL, being the limit value 500 cfu/mL.

Conforms Bacterial account (Wilson), less than 2.4 NMP/100 mL, the limit value being less than or equal 3.

Termotolerant Coliforms account (E.Coli), not present, the limit value being the absence in 100 mL.

Pseudomonas aeruginosa, absence, the limit value being absence in 100 mL. pH: 7,55,

specific conductivity: 60 umho/cm.

Salts in solution (105° C.): 45 mg/L. Muddiness: less than NTU.

Color: 2,5 c.u.

| | mEq/L | Mg/L |
|---|---|---|
| Anions | | |
| (CaCO3) | — | 22.5 |
| (CO₃²⁻) | — | 0 |
| (HCO₃⁻) | 0.45 | 27 |
| (Cl⁻) | 0.15 | 5 |
| (SO₄⁻) | — | Less than 10 |
| Cations | | |
| (CaCO₃) | — | 25 |
| (Ca⁺⁺) | 0.40 | 8 |
| (Mg⁺⁺) | 0.10 | 1.2 |
| (Na⁺) | 0.20 | 5 |
| (K⁺) | — | Less than 0.5 |

| | |
|---|---|
| mEq/mL | Mg/L |

| | | |
|---|---|---|
| Organic materia | — | 15 |
| (NO³⁻) | — | 1.6 |
| (NO²⁻) | — | Less than 0.05 |
| (PO₄³⁻) | — | Less than 0.1 |
| Iron | — | Less than 0.1 |
| Fluorure | — | Less than 0.1 |
| Arsenic | — | Less than 0.05 |

The invention may be better understood with reference to the following examples which are not limitative or restrictive of the scope of protection. On the contrary, it must be clearly understood that many other embodiments, modifications and alterations equivalent to the elements of the invention may be suggested by persons skilled in the art after reading the present description, without departing from the spirit of the present invention and/or the scope of the appended claims.

### EXAMPLES

An herbal composition according to the invention has been obtained by:

i) mixing 0.85 grams of the Artemisa absinthium L. and 1.15 grams of said Ruta graveolens L. and 1.5 liters of a mineral water according to the above specifications, with the mineral water at 100° C. and maintained at such temperature for 15 seconds, to form a mixture,

ii) permitting the mixture to rest for obtaining an infusion and decanting the herbs, and

iii) filtering the infusion to obtain a filtered solution.

The proportions of 0.85 grams of the Artemisa absinthium L., 1.15 grams of said Ruta graveolens L. and 1.5 liters of solvent, preferably mineral water, have been maintained for all the tests and these proportions are within the scope of the invention.

A preferred dose comprises 150 cm3 of infusion, therefore the contents of the components are 0.085 grams of Ajenjo and 0.15 grams of Ruda in 150 cm3 of mineral water.

Several patients were treated with the inventive composition and an important number of cases were analyzed as follows: Cancer (60 cases), Hepatitis C (7 cases), Pengifoide Buloso (1 case in Chile) and HIV (12 cases).

The treatment consisted in the administration of daily doses of 150 cm.3 of the infusion during the time of the treatment. The average time of the treatment was about 3 months and in all cases the final result was the complete cure of the disease with the total disappearance of the symptomatology. It's very important to remark that the herbal composition of the present invention do not seems to present side effects or unexpected reactions in the patients under treatment.

While preferred embodiments of the present invention have been illustrated and described, it will be obvious to those skilled in the art that various changes and modifications may be made therein without departing from the scope of the invention as defined in the appended claims.

I claim:

1. An herbal composition for the treatment of a patient afflicted of at least one of the following diseases, HIV/AIDS, cancer, hepatitis C and Pengifoide buloso, the composition comprising:
a mixture of herbs Artemisa absinthium L. and Ruta graveolens L. forming an infusion mixture in a solvent.

2. The herbal composition of claim 1, wherein said solvent is water.

3. The herbal composition of claim 2, wherein said water is mineral water.

4. The herbal composition of claim 3, comprising said Artemisa absinthium L., said Ruta graveolens L. and the mineral water, in the following proportion 0.85 grams of said Artemisa absinthium L. and 1.15 grams of said Ruta graveolens L. per 1.5 liter of the mineral water.

5. A method for preparing an herbal composition for the treatment of a patient afflicted of at least one of the following diseases, HIV/AIDS, cancer, hepatitis C and Pengifoide buloso, wherein the composition comprises:
a mixture of herbs Artemisa absinthium L. and Ruta graveolens L. forming an infusion mixture in a solvent,
the method comprising the steps of:
i) mixing the Artemisa absinthium L. and the Ruta graveolens L. in the solvent at 100° C. for 15 seconds, to form a mixture and in a proportion equivalent to 0.85 grams of the Artemisa absinthium L., 1.15 grams of the Ruta graveolens L. and 1.5 liters of the solvent,
ii) permitting the mixture to rest for obtaining an infusion and decanting the herbs, and
iii) filtering the infusion to obtain a filtered solution.
